(19) United States
(12) Patent Application Publication
(10) Pub. No.: US 2024/0288360 A1
(43) Pub. Date: Aug. 29, 2024

(54) APPARATUS FOR DETECTION OF ANALYTES

(71) Applicant: Nanopath Inc., North Cambridge, MA (US)

(72) Inventors: Alison Burklund, Cambridge, MA (US); Amogha Tadimety, Cambridge, MA (US); David Wolf, Cambridge, MA (US); Doug Sabin, Cambridge, MA (US); Travis Colosi, Cambridge, MA (US)

(21) Appl. No.: 18/689,840

(22) PCT Filed: Sep. 6, 2022
(86) PCT No.: PCT/US2022/075993

§ 371 Date: Mar. 6, 2024

Related U.S. Application Data
(60) Provisional application No. 63/241,356, filed on Sep. 7, 2021.

(51) Int. Cl. G01N 21/25; G01N 33/53

(52) U.S. Cl. CPC G01N 21/255; G01N 33/5308; G01N 2021/258; G01N 2201/021; G01N 2201/0636

(57) ABSTRACT

Provided herein is an apparatus for the detection of analytes. In some embodiments, the analyte to be detected consists of nucleotide sequences. In some embodiments, the analytes are detected in liquid solutions. In some embodiments, the apparatus comprises a stage, a light source, a spectrometer, and a lens assembly.

### CROSS-REFERENCE TO RELATED APPLICATIONS

The present application claims the benefit of U.S. Provisional Application No. 63/241,356, filed Sep. 7, 2021, which is hereby incorporated by reference in its entirety.

### BACKGROUND

### Field

The present disclosure generally relates to the detection or sensing of various materials, including biological and chemical substances. More specifically, provided herein is an apparatus and a method for the detection of nucleic acids.

### Description of the Related Art

For biomedical research, clinical diagnostics, environmental testing, and other related fields, it is beneficial to have apparatuses and systems for detecting analytes, such as biomolecules and chemical substances with high accuracy, sensitivity, specificity, reproducibility, and ease of use. For example, having fast, rapid, and accurate tests for detecting certain analytes in a biological sample may aid in clinical diagnosis contexts, and assist physicians in determining optimal treatment regimens.

One class of biomolecules that share a strong causal relationship to a disease state are nucleotides—detection of certain nucleotide sequences may implicate or confirm a clinical diagnosis. However, observation, detection, or otherwise analyzing nucleotides or nucleotide sequences in an efficient manner from primary patient samples has been stymied in clinical contexts for a variety of reasons. Therefore, an easy-to-operate analyte detection system or apparatus would provide tremendous benefit in a clinical setting.

### SUMMARY

Disclosed herein is an apparatus for use in detecting a variety of constituent analytes, such as biomolecules and chemical substances, within a sample, for example, polynucleotides within a liquid sample.

The systems, devices, kits, and methods disclosed herein each have several aspects, no single one of which is solely responsible for their desirable attributes. Without limiting the scope of the claims, some prominent features will be discussed herein. Numerous other examples are also contemplated, including examples that have fewer, additional, and/or different components, steps, features, objects, benefits, and advantages. The components, aspects, and steps may also be arranged and ordered differently. It is to be understood that any feature of the device and/or the apparatus disclosed herein may be combined together in any desirable manner and/or configuration. Further, it is to be understood that any features of the method of using the device may be combined together in any desirable manner. Moreover, it is to be understood that any combination of features of this method and/or of the device and/or of the array may be used together, and/or may be combined with any of the examples disclosed herein. Still further, it is to be understood that any feature or combination of features of any of the devices and/or of the arrays and/or of any of the methods may be combined together in any desirable manner, and/or may be combined with any of the examples disclosed herein.

In some embodiments, provided herein is an apparatus for spectroscopically determining the presence of a target analyte. The apparatus comprises a stage, a light source, a spectrometer, and a lens assembly. The stage comprises an actuator arm and a sample holder. In some embodiments, the sample holder is configured to receive microscope slides. In some embodiments, the microscope slides are 25 mm×75 mm. In some embodiments, the microscope slides are 1 mm thick. In some embodiments, the slides further feature a 1-2 mm thick PDMS layer with wells. In some embodiments, the microscope slide further features a coverslip. In some embodiments, the coverslip is about 170 μm. In some embodiments, the sample holder is configured to receive microfluidic devices, including but not limited to cartridges, cassettes, or modules designed to process fluid or solid samples. In some embodiments, multiple sensor sites are present on the surface of the microfluidic device or devices, the multiple sensor sites comprising populations of immobilized metallic nanoparticles. In some embodiments, the actuator arm has one or more degrees of articulation. In some embodiments, the light source is configured to emit a specific wavelength. In some embodiments, the light source is configured to emit a series of specific wavelengths. In some embodiments, the light source is configured to emit a series of specific wavelengths at varying intensities and durations. In some embodiments, the light source is configured to emit white light. In some embodiments, the lens assembly comprises a focusing element, an optic element, and at least one mirror. In some embodiments, the mirror or plurality of mirrors may be concave mirrors. In some embodiments, the mirror or plurality of mirrors may be parabolic. In some embodiments, the focusing element adjusts a focus plane of the optic element. In some embodiment, light emitted by the light source is reflected by the mirror or a plurality of mirrors. In some embodiments, the light path of the apparatus is determined wholly or in part by the direction of the mirror or plurality of mirrors. In some embodiments, the spectrometer is configured to intercept light emitted by the light source. In some embodiments, the spectrometer is configured to collect data regarding absorbance. In some embodiments, the spectrometer is configured to collect data regarding transmittance. In some embodiments, the spectrometer is configured to collect data regarding extinction. In some embodiments, the spectrometer is configured to collect data, including full spectra at defined wavelengths, when target analytes combine with metallic nanoparticles. In some embodiments, the spectrometer is configured to collect data, including wavelength shifts, when target analytes combine or are associated with metallic nanoparticles, including gold nanoparticles. In some embodiments, the spectrometer is configured to collect data regarding physical properties of nanoparticles. In some embodiments, the wavelength range of the spectrometer is between 500 nm and 1000 nm. In some embodiments, the stage and lens assembly will interact to optimize positioning and focusing of samples, including moving samples or sensor spots into an intersection position with a light beam. In some embodiments, the spectrometer will move to optimize positioning and focusing of samples or sensor spots.

In some embodiments, the apparatus is configured to generate an output. In some embodiments, the output is determined by absorbance, transmittance, or extinction measured by the spectrometer. In some embodiments, the spectrometer measures any wavelength shift due to the binding of metallic nanoparticles to any number of analyte species. In some embodiments, the binding of metallic nanoparticles to any number of analyte species changes the refractive index. In some embodiments, the change in refractive index is a result of surface plasmon resonance or other resonant oscillation events. In some embodiments, the output comprises absorbance, transmittance, or extinction data from a preset wavelength. In some embodiments, the output comprises absorbance, transmittance, or extinction data from a set of preset wavelengths.

In some embodiments of the present disclosure, a user will configure the apparatus using a graphical user interface (GUI). In some embodiments, the apparatus output will be displayed to a user using the GUI. In some embodiments, the user may configure the apparatus to determine spectrometer integration time, measurement location, and algorithm settings.

It should be appreciated that all combinations of the foregoing concepts and additional concepts discussed in greater detail below are contemplated as being part of the inventive subject matter disclosed herein and may be used to achieve the benefits and advantages described herein.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1 illustrates an embodiment of the present disclosure.

FIG. 2 illustrates wavelength and intensity of embodiments of the light source.

FIG. 3 illustrates actuator positions in an embodiment of the present disclosure.

FIG. 4 illustrates actuator positions with relation to a spectrometer sensor of the present disclosure.

FIG. 5 illustrates GUI inputs and prompts along with apparatus status in an embodiment of the present disclosure.

FIG. 6 illustrates GUI inputs and prompts along with apparatus status in an embodiment of the present disclosure.

FIG. 7 illustrates GUI screen progression according to an embodiment of the present disclosure.

FIG. 8 illustrates cable inputs according to an embodiment of the present disclosure.

FIG. 9 illustrates a light source and spectrometer according to an embodiment of the present disclosure.

FIG. 10 illustrates an optic path and sensor assembly according to an embodiment of the present disclosure.

FIG. 11 illustrates a sample cassette and sample chip according to an embodiment of the present disclosure.

FIGS. 12-14 show GUI menus according to an embodiment of the present disclosure.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

### Definitions

All technical and scientific terms used herein have the same meaning as commonly understood to one of ordinary skill in the art to which this disclosure belongs unless clearly indicated otherwise.

As used herein, the singular forms “a”, “and”, and “the” include plural referents unless the context clearly dictates otherwise. Thus, for example, reference to “a sequence” may include a plurality of such sequences, and so forth.

The terms comprising, including, containing and various forms of these terms are synonymous with each other and are meant to be equally broad. Moreover, unless explicitly stated to the contrary, examples comprising, including, or having an element or a plurality of elements having a particular property may include additional elements, whether or not the additional elements have that property.

As used herein, a spectrometer refers to any scientific instrument used to separate and measure spectral components of a physical phenomenon. In the context of optical spectrometers, an optical spectrometer is capable of measuring the spectrum of light and measure the intensity of said light as a function of wavelength or frequency. Light detected by a spectrometer can consist of a continuous spectrum, an emission spectrum, a transmittance spectrum, an extinction spectrum, or absorption spectrum.

### Introduction

The present disclosure generally relates to an apparatus and system for analyzing samples using optical spectroscopy. In particular, the present disclosure may analyze a series of samples, in some instances liquid samples, and detect the presence or absence of various analytes based on wavelength shifts generated when a target analyte interacts or binds with metal nanoparticles. In some embodiments, the metal nanoparticles are immobilized on a surface, with specific populations of metal nanoparticles at predetermined spots or sensors on the surface. Based on the specific wavelength shift generated and measured by a spectrometer according to various embodiments of the present disclosure, the presence of certain nucleotide sequences corresponding to specific or several disease states may be detected.

In some embodiments, provided herein is an apparatus for spectroscopically determining the presence of a target analyte. The apparatus comprises a stage, a light source, a spectrometer, and a lens assembly. The stage comprises an actuator arm and a sample holder. In some embodiments, the sample holder is configured to receive microscope slides. In some embodiments, the microscope slides are 25 mm×75 mm. In some embodiments, the microscope slides are 1 mm thick. In some embodiments, the slides further feature a 1-2 mm thick PDMS layer with wells. In some embodiments, the microscope slide further features a coverslip. In some embodiments, the coverslip is about 170 μm. In some embodiments, the sample holder is configured to receive microfluidic devices, including but not limited to cartridges, cassettes, or modules designed to process fluid or solid samples. In some embodiments, multiple sensor sites are present on the surface of the microfluidic device or devices, the multiple sensor sites comprising populations of immobilized metallic nanoparticles. In some embodiments, the actuator arm has one or more degrees of articulation. In some embodiments, the light source is configured to emit a specific wavelength. In some embodiments, the light source is configured to emit a series of specific wavelengths. In some embodiments, the light source is configured to emit a series of specific wavelengths at varying intensities and durations. In some embodiments, the light source is configured to emit white light. In some embodiments, the lens assembly comprises a focusing element, an optic element, and at least one mirror. In some embodiments, the mirror or plurality of mirrors may be concave mirrors. In some embodiments, the mirror or plurality of mirrors may be parabolic. In some embodiments, the focusing element adjusts a focus plane of the optic element. In some embodiment, light emitted by the light source is reflected by the mirror or a plurality of mirrors. In some embodiments, the light path of the apparatus is determined wholly or in part by the direction of the mirror or plurality of mirrors. In some embodiments, the spectrometer is configured to intercept light emitted by the light source. In some embodiments, the spectrometer is configured to collect data regarding absorbance. In some embodiments, the spectrometer is configured to collect data regarding transmittance. In some embodiments, the spectrometer is configured to collect data regarding extinction. In some embodiments, the spectrometer is configured to collect data, including full spectra at defined wavelengths, when target analytes combine with metallic nanoparticles. In some embodiments, the spectrometer is configured to collect data, including wavelength shifts, when target analytes combine or are associated with metallic nanoparticles, including gold nanoparticles. In some embodiments, the spectrometer is configured to collect data regarding physical properties of nanoparticles. In some embodiments, the wavelength range of the spectrometer is between 500 nm and 1000 nm. In some embodiments, the stage and lens assembly will interact to optimize positioning and focusing of samples, including moving samples or sensor spots into an intersection position with a light beam. In some embodiments, the spectrometer will move to optimize positioning and focusing of samples or sensor spots.

In some embodiments, presented herein is an apparatus for detecting one or more analytes in one or more sensors. The apparatus comprising a light source, a spectrometer, and a lens assembly, wherein the lens assembly comprises a focusing element and a mirror, wherein the light source is configured to excite electrons within the one or more sensor(s), wherein the spectrometer is configured to detect surface plasmon resonance events. In some embodiments, the spectrometer is configured to detect the one or more analytes. In some embodiments, the one or more analytes comprise nucleic acids, cell free nucleic acids, DNA, RNA, miRNA, oligonucleotides, peptide nucleic acids, proteins, or cells. In some embodiments, each sensor of the one or more sensors comprises metallic nanoparticles. In some embodiments, the metallic nanoparticles bind to one or more analytes. In some embodiments, the binding of metallic nanoparticles to one or more analytes generates a change in the refractive index. In some embodiments, the change in refractive index is due to surface plasmon resonance (SPR). In some embodiments, the apparatus further comprises one or more mirrors to direct a light path emitted by the light source. In some embodiments, the apparatus further comprises a stage and an articulating arm, wherein the articulating arm is mechanically linked to the stage, wherein the articulating arm is configured to move the stage in multiple dimensions to intersect with the light path at one or more predetermined points in space. In some embodiments, the metallic nanoparticles are immobilized on a surface. In some embodiments, the surface is atop the stage. In some embodiments, the surface is in a sample holding device, including but not limited to a cartridges, cassettes, or modules designed to process fluid or solid samples. In some embodiments, the surface is transparent.

In some embodiments, a method for detecting one or more analytes in one or more samples is described. In some embodiments, the method comprises loading one or more samples onto a surface containing one or more sensors, then placing the surface into an apparatus comprising a stage, a light source, and a spectrometer, wherein the one or more samples are atop one or more surfaces, each surface of the one or more surfaces containing one or more sensors comprising immobilized metallic particles. The method further comprises exposing the surface to a light from the light source at a series of wavelengths, and measuring absorbance, transmittance, or extinction data of the immobilized metallic particles. After exposing the surface to a light from the light source, the method also comprises measuring absorbance, transmittance, or extinction data of the immobilized metallic particles and comparing the absorption spectrum, transmission spectrum, or extinction spectrum of the immobilized metallic particles before and after exposure to an analyte of interest.

In some embodiments, the one or more analytes comprise nucleic acids, cell free nucleic acids, DNA, RNA, miRNA, oligonucleotides, peptide nucleic acids, proteins, or cells. In some embodiments, the one or more samples, one or more analytes, or surface is first exposed a to a thermal, mechanical, chemical, or biological treatment such that cells are lysed. In some embodiments, the analytes are concentrated via an enrichment or filtration step. In some embodiments, the filtration step can comprise any number of tangential flow or ultrafiltration steps. In some embodiments, the enrichment step can pool multiple cell populations or cell population derived materials. In some embodiments, the one or more analytes comprise bacteria, virus, human cells, and/or respective genetic material of the foregoing. In some embodiments, the comparing step comprises observing an optical peak shift when bacterial, virus, human cells, or the respective genetic material thereof, are present.

In some embodiments, a method for detecting one or more analytes in a plurality of sensors is described. The method comprises loading the plurality of sensors onto an apparatus comprising a stage, a light source, an articulating arm, and a spectrometer, wherein the plurality of sensors each comprise a surface comprising immobilized metallic particles, wherein each sensor in the plurality of sensors is physically isolated from every other sensor in the plurality of sensors. In some embodiments, the method further comprises moving, by the articulating arm, the stage such that a sensor in the plurality of sensors intersects a beam path originating from the light source. Next, the method comprises emitting, from the light source, a light at a series of wavelengths on to the surface of the sensors, the light traveling along the beam path, and capturing, by the spectrometer, absorbance, transmittance, or extinction data of the surface. The method further recites comparing the absorption spectrum, transmission spectrum, or extinction spectrum of the sensors with a reference spectrum.

In some embodiments, the one or more analytes comprise nucleic acids, cell free nucleic acids, DNA, RNA, miRNA, oligonucleotides, peptide nucleic acids, proteins, or cells. In some embodiments, the one or more samples, one or more analytes, or surface is first exposed a to a thermal, mechanical, chemical, or biological treatment such that cells are lysed. In some embodiments, the analytes are concentrated via an enrichment or filtration step. In some embodiments, the filtration step can comprise any number of tangential flow or ultrafiltration steps. In some embodiments, the enrichment step can pool multiple cell populations or cell population derived materials. In some embodiments, the one or more analytes comprise bacteria, virus, human cells, and/or respective genetic material of the foregoing. In some embodiments, the comparing step comprises observing an optical peak shift when bacterial, virus, human cells, or the respective genetic material thereof, are present.

### Operation

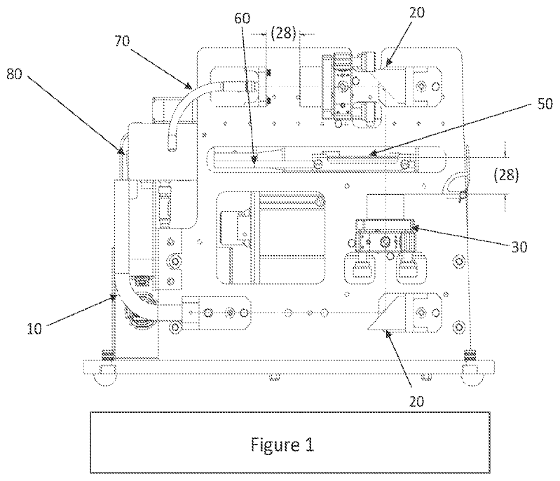

FIG. 1 illustrates an embodiment of apparatus for detection of analyte as described in the present disclosure, illustrating a light path and optical design for light emitted at light source 10. In the embodiment, light is emitted at light source 10, reflecting off mirror 20 and reflected from precision mirror 30. Light passing through precision mirror 30 then passes through stage 50, where the light is then reflected by mirror 40 and directed towards fiber optical cable 70, where the light path ends at spectrometer 80. In some embodiments, translating arm 60 is configured to move the nest 50, either to extend the stage for loading samples, or for altering the specific sample or position that is being analyzed by the spectrometer. In some embodiments, precision mirror 30 further consists of optical elements configured to focus and direct the light beam emitted by light source 10. These additional embodiments include two lenses that are within the optical stage, one to focus and adjust the light before hitting the sample, the other to focus the light that is passed through the spectrometer into the sample. Light passing through a sample loaded into stage 50 generates absorbance data which is collected by spectrometer 80. The wavelength shift generated when the analyte is combined with metallic nanoparticles may also be collected.

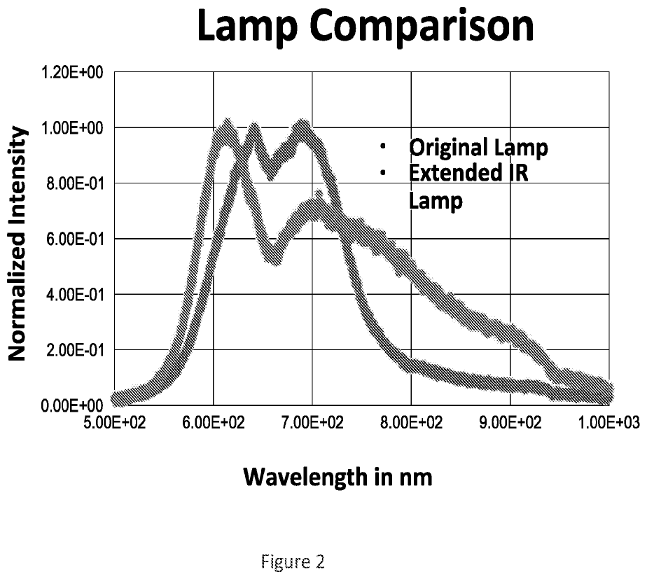

FIG. 2 illustrates an embodiment of the light source comparison regarding intensity as a function of wavelength. In some embodiments, the light source may be referred to as a lamp. It may be advantageous to have a light source with the capability to emit light in a wider range outside of the normal visible spectrum. In some embodiments, the collection of absorbance properties at higher near-infrared and infrared wavelengths is provided.

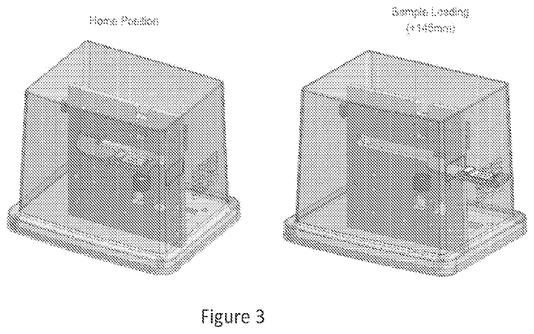

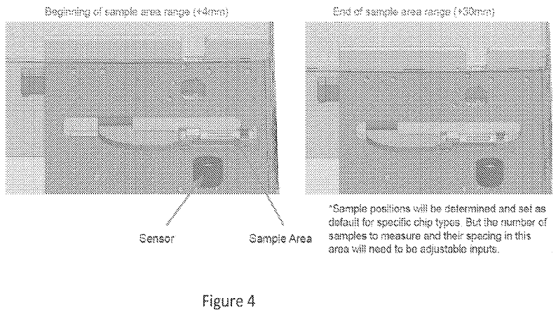

FIGS. 3 and 4 illustrate an embodiment of the present disclosure, wherein the articulating arm is operable connected to the stage. In some embodiments, the stage may further comprise a sample area. In FIG. 3, the articulating arm extends the stage to a position outside the assembly enclosure for sample loading onto the stage and sample area. Once loaded, the articulating arm retracts, moving the stage and the sample area within the enclosure. FIG. 4 illustrates a sample area and stage within the apparatus. In some embodiments, the sample area is positioned in between the light path generated in some embodiments of the present disclosure. In some embodiments, the articulating arm is capable of moving the stage to position the sample area in the light path. In some embodiments, multiple samples are loaded onto a sample area. In some embodiments, movement of the sample area allows for a new sample to be set in the light path.

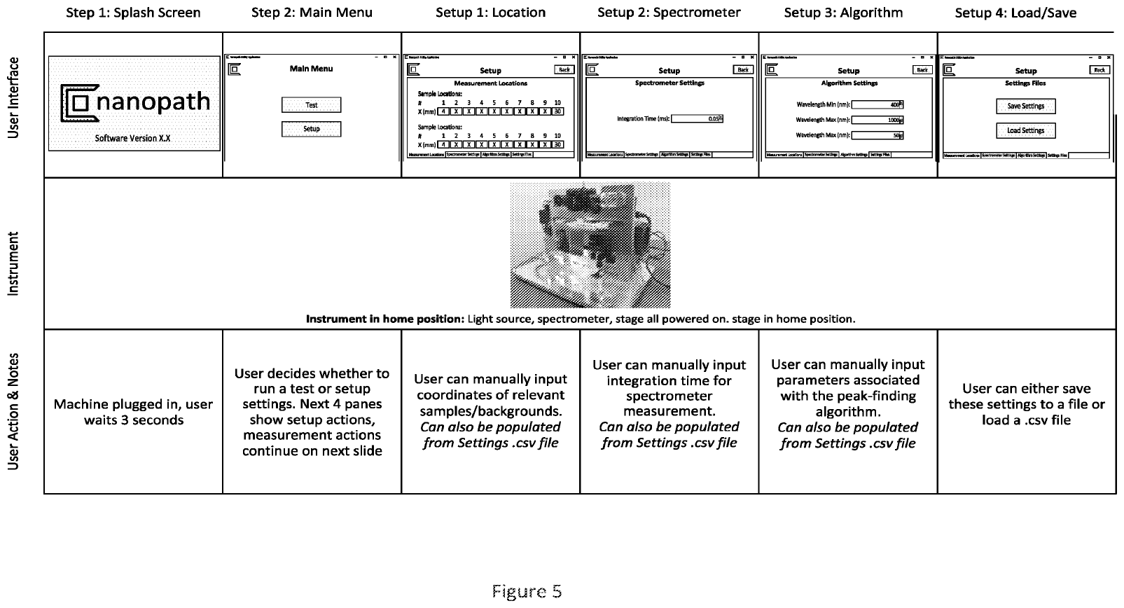

FIG. 5 illustrates an embodiment of a GUI of the present disclosure, a graphic user interface (GUI) wherein a user may input settings and select options that alter the Apparatus' automated sample processing and analysis algorithm. Step 1 illustrates a splash screen of the present disclosure. Step 2 allows a user to choose whether to run a test, or to select individualized parameters based on their experimental design. Setup 1 allows a user to select and input coordinates of relevant samples and controls. Setup 2 configures integration times for spectrometer measurements, while Setup 3 allows users to manually input parameters associated with a peak-finding algorithm. Lastly, Setup 4 allows users to either save or load settings from a file.

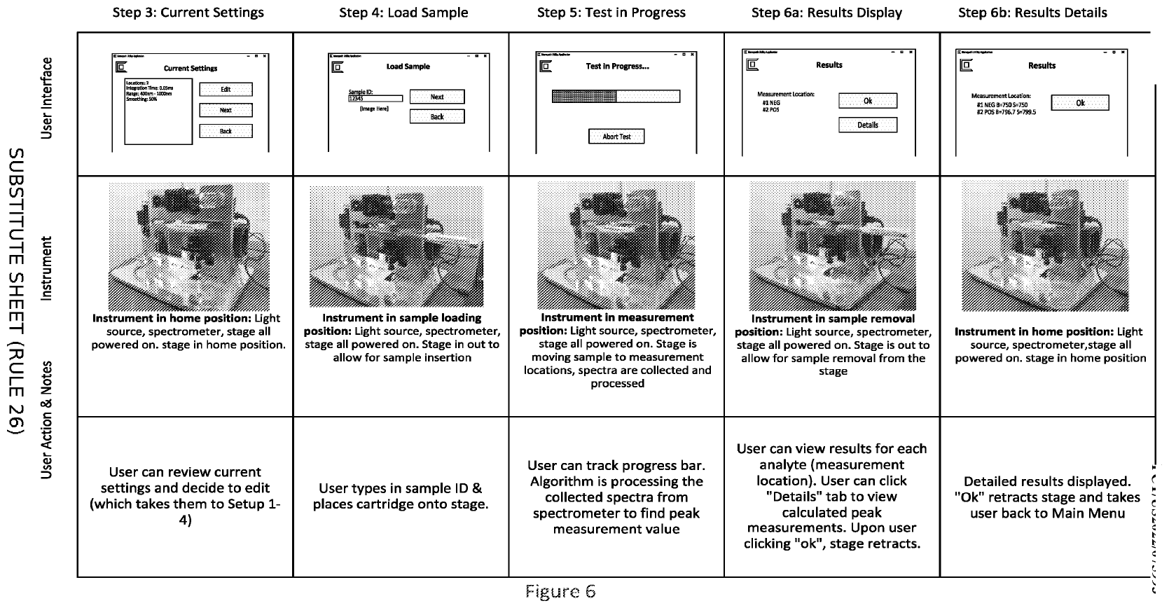

FIG. 6 illustrates an embodiment of the present disclosure wherein a user selects desired settings for the apparatus, runs the apparatus to generate an output, and reviews the output results.

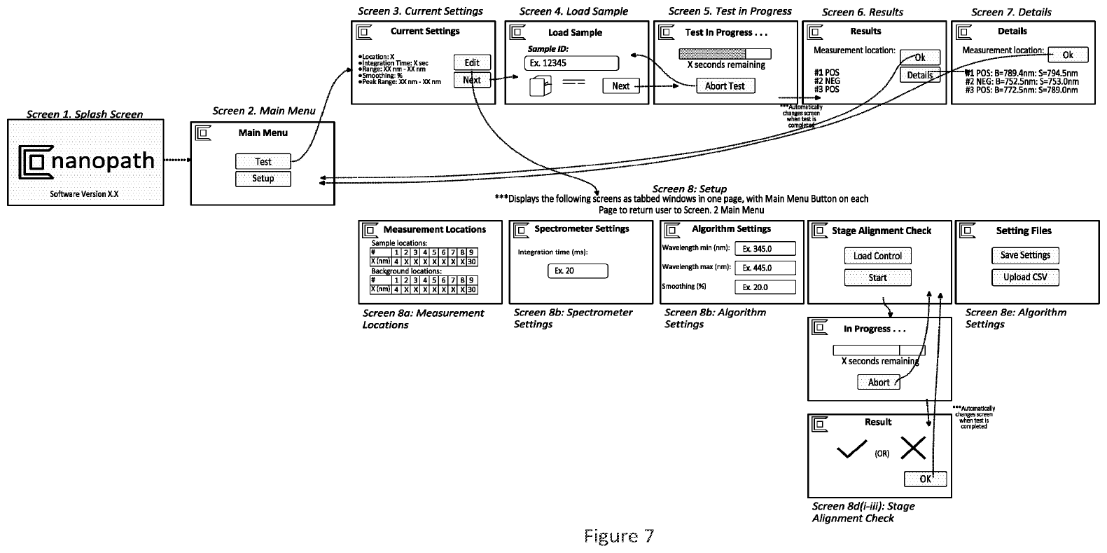

FIG. 7 illustrates an embodiment of the present disclosure, wherein user menu options direct to subsequent or prior menu screens based on user input. In some embodiments, screen 1 progresses to screen 2, which can load either screen 3 or screen 8, depending on whether a user selects “Test” or “Setup”. A user at screen 3 can progress to either screen 4 or screen 8, depending on which menu option is chosen. Screen 4 progresses to screen 5, where a user can then abort and return to screen 4, or allow the test to run to completion and view results on screen 6. Screen 6 allows for details of the run to be viewed on screen 7.

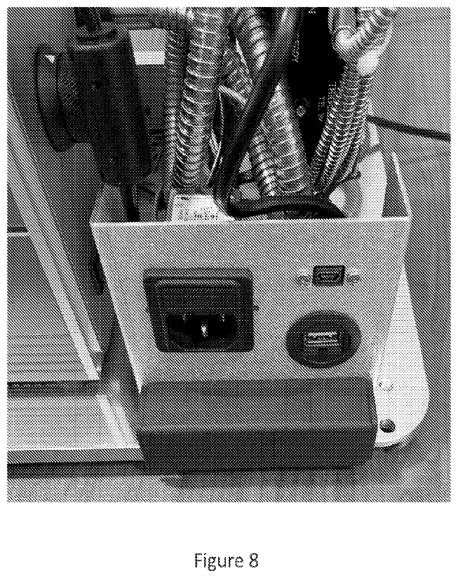

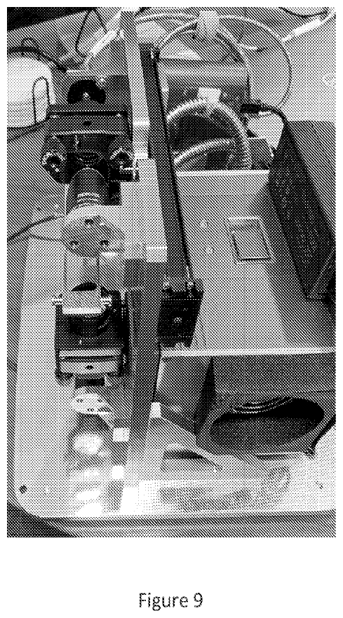

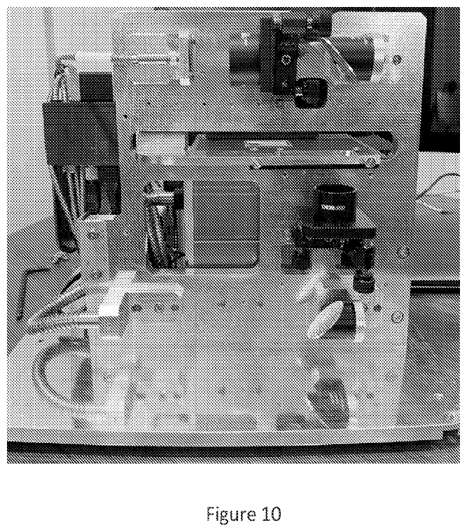

FIG. 8 illustrates a series of cable inputs in an embodiment of the present disclosure. These cable inputs may include a power input, and at least one data output port. In some embodiments, a plurality of data output ports may be present. FIG. 9 illustrates a light source and spectrometer assembly of the present disclosure. Additionally, FIG. 10 illustrates an optical path and light path according to the present disclosure.

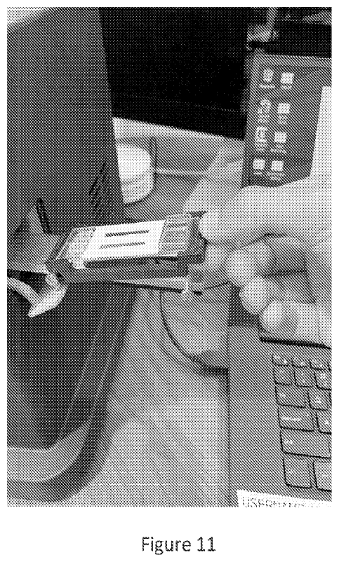

FIG. 11 illustrates the placement of a sample cassette and sample chip on the stage of the apparatus of the present disclosure. A sample, such as a biological sample or an environmental sample, is introduced into the sample chip. The sample chip comprises a region with plasmonic nanomaterials, such as metal nanoparticles, configured to bind at least one analyte to be detected. Once the sample chip and the sample cassette are position onto the stage, the stage retracts into the housing of the analyte detecting device. The absorption spectrum is collected while directing the light path through the plasmonic nanoparticle region.

The unique physical properties of plasmonic nanomaterials, such as metal nanoparticles, allow for the convenient detection of certain analytes through surface plasmon resonance (SPR) or localized surface plasmon resonance (LSPR). Analysis of absorbance when the analyte couples to the plasmonic nanoparticles can quickly and effectively allow the diagnosis of certain disease states.

In some embodiments, provided herein is a method relating to the detection of analytes. In some embodiments, the method may comprise loading a sample onto an apparatus comprising a stage, a light source, a spectrometer, and a lens assembly, wherein the sample comprises a surface including immobilized metallic particles and analyte complexes. Then emitting, by the light source, a light, wherein the light is configured to excite electrons at specific wavelengths and capturing, by the spectrometer, a wavelength shift, and generating absorbance, transmittance, or extinction data, wherein the apparatus is configured to direct the light to follow a light path.

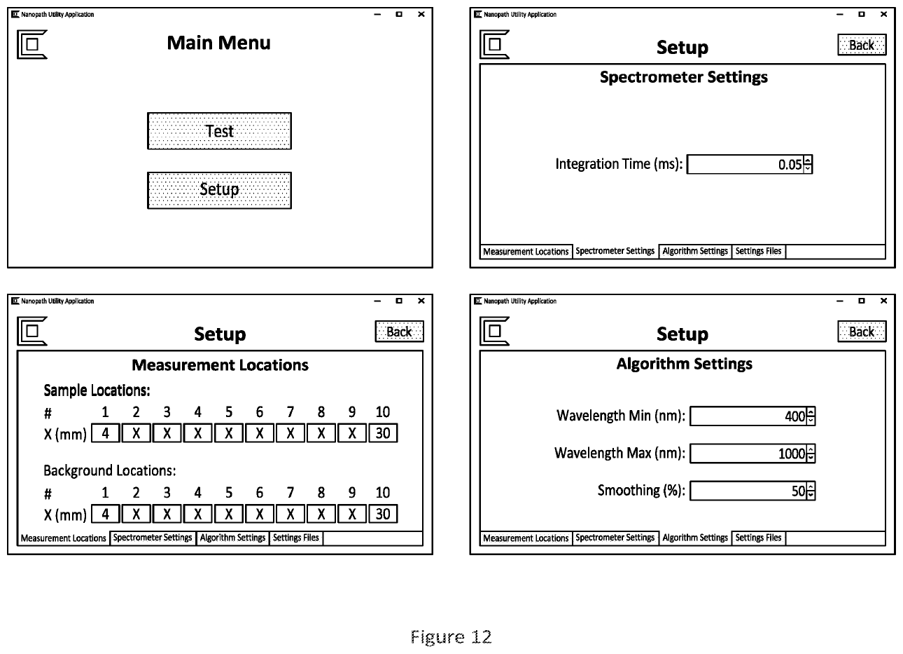

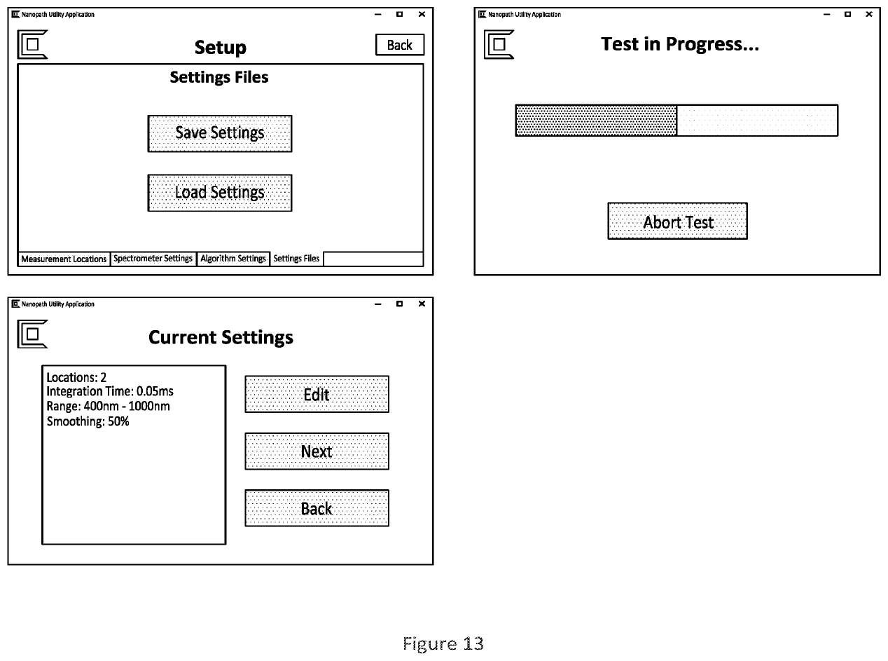

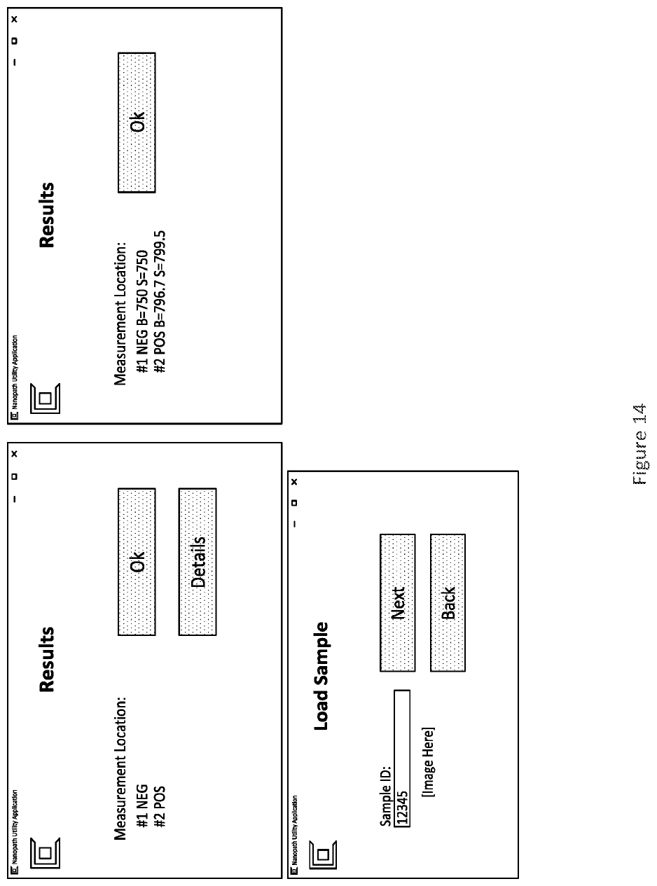

FIGS. 12-14 show a series of GUI menu, setup, measurement, and output displays of the present disclosure.

While certain examples have been described, these examples have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. In particular, elements presented relating to GUI elements or displays to a user may be presented in any particular order to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure. Also, the separation of various system components in the implementations described above should not be understood as requiring such separation in all implementations, and it should be understood that the described components and systems can generally be integrated together in a single product or packaged into multiple products. For example, any of the components for an light path system described herein can be provided separately, or integrated together (e.g., packaged together, or attached together) to form a analyte analysis system.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims. All references cited herein are incorporated by reference in their entirety.

The terminology used in the description presented herein is not intended to be interpreted in any limited or restrictive manner and unless otherwise indicated refers to the ordinary meaning as would be understood by one of ordinary skill in the art in view of the specification. Furthermore, embodiments may comprise, consist of, consist essentially of, several novel features, no single one of which is solely responsible for its desirable attributes or is believed to be essential to practicing the embodiments herein described. As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

Although this disclosure is in the context of certain embodiments and examples, those of ordinary skill in the art will understand that the present disclosure extends beyond the specifically disclosed embodiments to other alternative embodiments and/or uses of the embodiments and obvious modifications and equivalents thereof. In addition, while several variations of the embodiments have been shown and described in detail, other modifications, which are within the scope of this disclosure, will be readily apparent to those of ordinary skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes or embodiments of the disclosure. Thus, it is intended that the scope of the present disclosure herein disclosed should not be limited by the particular disclosed embodiments described above.

1. An apparatus for detecting one or more analyte(s) in one or more sensor(s), comprising:
a light source;
a spectrometer; and
a lens assembly, wherein the lens assembly comprises a focusing element and a mirror, wherein the light source is configured to excite electrons within the one or more sensor(s), wherein the spectrometer is configured to detect surface plasmon resonance events.

2. The apparatus of claim 1, wherein the spectrometer is configured to detect the one or more analyte(s).

3. The apparatus of claim 1, wherein the one or more analyte(s) comprise nucleic acids, cell free nucleic acids, DNAs, RNAs, miRNAs, oligonucleotides, peptide nucleic acids, proteins, or cells.

4. The apparatus of claim 1, wherein each sensor of the one or more sensor(s) further comprise metallic nanoparticles.

5. The apparatus of claim 4, wherein the metallic nanoparticles bind to the one or more analyte(s).

6. The apparatus of claim 1, wherein the apparatus further comprises one or more mirror(s) to direct a light path emitted by the light source.

7. The apparatus of claim 6, further comprising a stage and an articulating arm, wherein the articulating arm is mechanically linked to the stage, wherein the articulating arm is configured to move the stage in multiple dimensions to intersect with the light path at one or more predetermined points in space.

8. The apparatus of claim 4, wherein the metallic nanoparticles are immobilized on a surface.

9. The apparatus of claim 8, wherein the surface is transparent.

10. A method for detecting one or more analyte(s) in one or more sample(s), comprising:
loading the one or more sample(s) onto a surface containing one or more sensor(s),
placing the surface into an apparatus comprising a stage, a light source, and a spectrometer, wherein the one or more sample(s) are atop one or more surfaces, each surface of the one or more surfaces containing one or more sensor(s) comprising immobilized metallic particles;
exposing the surface to a light from the light source at a series of wavelengths;
measuring absorbance, transmittance, or extinction data of the immobilized metallic particles; and
comparing the absorption spectrum, transmission spectrum, or extinction spectrum of the immobilized metallic particles before and after exposure to an analyte of interest.

11. The method of claim 10, wherein the one or more analyte(s) comprise nucleic acids, cell free nucleic acids, DNAs, RNAs, miRNAs, oligonucleotides, peptide nucleic acids, proteins, or cells.

12. The method of claim 10, where the one or more sample(s), one or more analyte(s), or surface is first exposed to a thermal, a mechanical, a chemical, or a biological treatment such that the cells are lysed.

13. The method of claim 10, where the analyte(s) is concentrated via an enrichment or filtration step.

14. The method of claim 10, wherein the one or more analyte(s) comprises bacteria, virus, human cell, and/or their respective genetic material.

15. The method of claim 10, wherein the comparing step comprises observing an optical peak shift when bacteria, virus, human cell, and/or their respective genetic material are present.

16. A method for detecting one or more analyte(s) in a plurality of sensors, comprising:
loading the plurality of sensors onto an apparatus comprising a stage, a light source, an articulating arm and a spectrometer, wherein the plurality of sensors each comprises a surface comprising immobilized metallic particles, wherein each sensor in the plurality of sensors is physically isolated from every other sensor in the plurality of sensors;
moving, by the articulating arm, the stage such that a sensor in the plurality of sensors intersects a beam path originating from the light source;
emitting, from the light source, a light at a series of wavelengths onto the surface of the sensors, traveling along the beam path;
capturing, by the spectrometer, absorbance, transmittance, or extinction data of the surface; and
comparing absorption spectrum, transmission spectrum, or extinction spectrum of the sensors with a reference spectrum.

17. The method of claim 16, wherein the one or more analyte(s) comprise nucleic acids, cell free nucleic acids, DNAs, RNAs, miRNAs, oligonucleotides, peptide nucleic acids, proteins, or cells.

18. The method of claim 16, where the one or more sample(s), one or more analyte(s), or surface is first exposed to a thermal, a mechanical, a chemical, or a biological treatment such that the cells are lysed.

19. The method of claim 16, where the analyte(s) is concentrated via an enrichment or filtration step.

20. The method of claim 16, wherein the reference spectrum is a baseline data of the metallic particles captured by the spectrometer prior to exposure and incubation with target analyte.

21. The method of claim 16, wherein the one or more analyte(s) comprises bacteria, virus, human cell and/or their respective genetic material.

22. The method of claim 16, wherein the comparing step comprises observing an optical peak shift when bacteria, virus, human cell and/or their respective genetic material are present.
